(19) United States
(12) Patent Application Publication
(10) Pub. No.: US 2021/0008354 A1
(43) Pub. Date: Jan. 14, 2021

(54) ANGIOPLASTY OF CALCIFIED ARTERIES

(71) Applicant: Imperial College of Sciencs,Technology and Medicine, London (GB)

(72) Inventor: Mohammad Reza Bhamanyar, London (GB)

(21) Appl. No.: 16/982,706

(22) PCT Filed: Sep. 21, 2019
(86) PCT No.: PCT/GB2019/050812

§ 371 Date: Sep. 21, 2020

(30) Foreign Application Priority Data
Mar. 22, 2018 (GB) 1804589.8

(51) Int. Cl. A61M 25/10

(52) U.S. Cl. CPC A61M 25/104; A61M 2205/0266; A61M 2025/109; A61M 25/1018

(57) ABSTRACT

A medical device comprises a catheter having a lumen extending between a distal end and a proximal end of the catheter and a balloon having an expandable interior volume coupled to the distal end of the catheter with its interior volume in fluid communication with the lumen of the catheter. A spring-loadable mechanical structure is disposed within the balloon configured to snap between first and second configurations upon actuation by an actuation mechanism. Upon actuation, the spring-loadable mechanical structure creates pressure pulses in fluid within the balloon interior volume which are transmitted to the walls of the balloon to disrupt calcified material or other hardened material within a vessel of the human or animal body in which the balloon is situated.

The present invention relates to methods and apparatus suitable for the disruption and/or disintegration of material that has built up on and/or in arterial walls, particularly though not exclusively for use during angioplasty procedures.

Coronary artery disease is caused by deposition of low-density lipoprotein (LDL) in the arterial wall, calcification and the subsequent formation of plaque. It is often treated by percutaneous coronary intervention (PCI), including Plain Old Balloon Angioplasty (POBA) as well as stenting and atherectomy. Coronary calcification restricts balloon infiltration (e.g. in balloon angioplasty) and increases procedural complexity and risk of complication including coronary perforation and dissection.

Existing methods used to address the challenges of calcification include so-called ‘cutting’ balloons (balloons fitted with blades) and rotational atherectomy where a rotating diamond coated burr is introduced and used to disrupt the calcification. Such techniques require highly experienced surgeons and can increase periprocedural complication rates.

A challenge with vascular mural calcification is that it can be hard and brittle. This combination can create the particular difficulties during percutaneous angioplasty referred to above. To overcome hardness may require the use of very high pressures during balloon inflation, whilst brittleness means that instead of plastic deformation occurring under the application of increasing pressure, a sudden failure may occur. It is this hard and brittle behaviour which makes traditional balloon dilation of calcified vessels difficult or risky (e.g. potentially leading to dissection or perforation as a result of sudden calcium fracturing at high inflation pressures).

However, it is the hardness and brittleness of calcium that can be exploited in this invention. Lack of plastic deformation in calcium results in fracture upon application of a large enough force. Mechanical impacts result in production of large forces over the short impulse time during which the energy is transferred from one object to the other. This principle can be used to disrupt vascular mural calcification by local application of a mechanical impulse of sufficient energy.

WO 2017/168145 describes an approach deploying a medical device which has a catheter with a lumen extending between a distal end and a proximal end of the catheter to an inflatable balloon at the distal end of the catheter. The lumen of the catheter is used to deliver, from a pressure pump at a proximal end of the catheter, a baseline fluid pressure to the balloon to inflate the balloon to a normal operating pressure for radial expansion of the balloon to engage the walls of a vessel in which the balloon is located. A pressure modulation source is used to apply pressure impulses to catheter lumen and thereby to the balloon walls. The pressure impulses are of short duration and are transmitted by the fluid within the catheter lumen and the balloon to the inflated surface of the balloon. Such percussive pressure impulses can produce significant force over a short time interval to the balloon, which is provided with an outer surface structure that is particularly configured to localise outward force of the balloon walls to hard hammer surfaces.

It is an object of the invention to provide one or more alternative ways to deliver such percussive forces to a balloon suitable for the break-up, disruption or disintegration of calcified material or other hardened material within vessels of the human or animal body. Such material may otherwise prevent or inhibit stenting procedures or passage of guidewires, catheters and other devices through the vessels.

According to one aspect, the present invention provides a medical device comprising:
a balloon having an expandable interior volume for coupling to the distal end of a catheter for a lumen of the catheter to be in fluid communication with the expandable interior volume of the balloon;
a spring-loadable mechanical structure within the balloon configured to snap between first and second configurations upon actuation by an actuation mechanism.

The medical device may further comprise a catheter having a lumen extending between a distal end and a proximal end of the catheter. The balloon may be coupled to the distal end of the catheter and have its interior volume in fluid communication with the lumen of the catheter. The actuation mechanism may comprise fluid pressure transmitted through the catheter. The medical device may include a shell disposed within the balloon. The shell may define an internal volume and an activation surface forming part of the shell. The activation surface may comprise the spring-loadable mechanical structure configured to snap between said first and second configurations. The first configuration of the activation surface may be a concave configuration and the second configuration may be a convex configuration. The lumen may comprise a first lumen communicating with the interior volume of the balloon and a second lumen communicating with the internal volume of the shell. The spring-loadable mechanical structure may be configured to store mechanical potential energy during a loading phase while it goes from a first configuration to an unstable mid-point which is impulsively released when the mechanical structure is forced to transition to the second configuration. The medical device may further include a means for applying fluid pressure to the interior volume of the balloon through the first lumen and a means for applying pressure pulses to the internal volume of the shell through the second lumen. The shell may comprise a hollow member with supporting wall portions and at least one flexible wall portion supported by the supporting wall portions. The flexible wall portion may have a flexibility substantially greater than the supporting wall portions and be configured to snap between the first and second configurations. The supporting wall portions may have a first thickness and the flexible wall portions may have a second thickness less than the first thickness. At least the spring-loadable mechanical structure may be formed of nickel, nickel alloy or of nickel-titanium alloy.

The actuation mechanism may comprise an electromechanical device. The electromechanical device may be located within the balloon and may be coupled to a control wire extending along a catheter to the balloon. The spring-loadable mechanical structure may comprise a thermally actuatable element. The spring-loadable mechanical structure may comprise a bimetallic element. The actuation mechanism may comprise an electrical heating element. The electrical heating element may form part of the bimetallic element.

The spring-loadable mechanical structure within the balloon may comprise a housing defining a cavity with at least one opening to the cavity and an elastic strip retained within the cavity and partially occluding the cavity opening to substantially restrict fluid flow into and out of the cavity via the opening. The elastic strip may define an activation surface configured to snap between first and second configurations, the first configuration comprising a concave configuration and the second configuration comprising a convex configuration. The medical device may further comprise an aperture in a wall of the housing, the aperture extending to the cavity to enable fluid pressure pulses to be delivered from outside the housing into the cavity to actuate the elastic strips between the first and second configurations.

According to another aspect, the invention provides a method of breaking up, disrupting or disintegrating calcified or other hardened material within vessels of the human or animal body comprising:
using a catheter to deploy a medical device into the vessel, the medical device comprising a balloon having an expandable interior volume coupled to the distal end of the catheter such that a lumen of the catheter is in fluid communication with the expandable interior volume of the balloon;
inflating the balloon within the vessel with a fluid to fill the expandable interior volume of the balloon;
actuating a spring-loadable mechanical structure within the balloon to snap between a first configuration and a second configuration using an actuation mechanism, to thereby create a pressure impulse in the fluid within the interior volume of the balloon.

The pressure impulse may generate cavitation in the fluid within the balloon.

Embodiments of the present invention will now be described by way of example and with reference to the accompanying drawings in which:

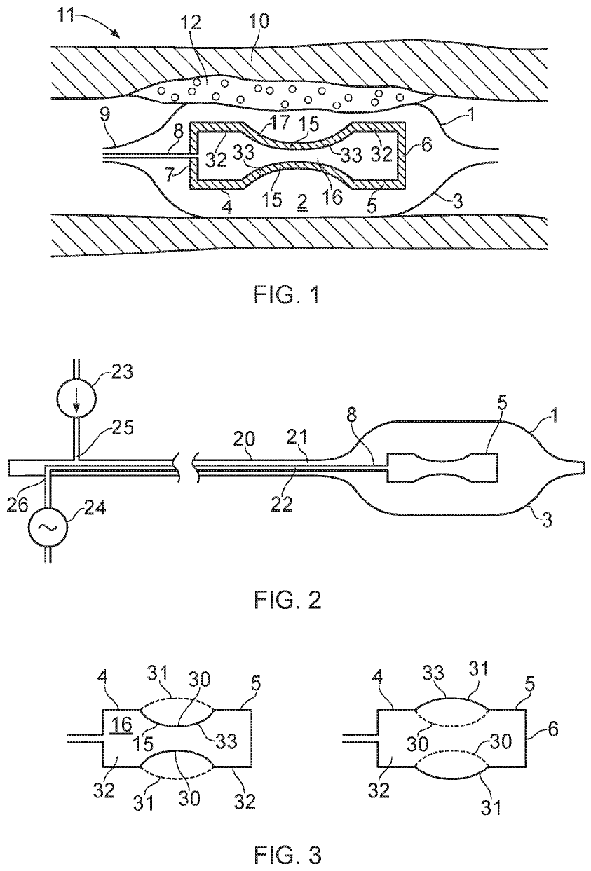

FIG. 1 shows a schematic cross-sectional side view of a balloon for a medical device, with the balloon incorporating a spring-loadable mechanical structure within its interior volume;

FIG. 2 shows a schematic cross-sectional side view of a medical device incorporating the balloon of FIG. 1;

FIG. 3 shows a schematic cross-sectional side view of the spring-loadable mechanical device of FIG. 1 illustrated in each of its two configurations;

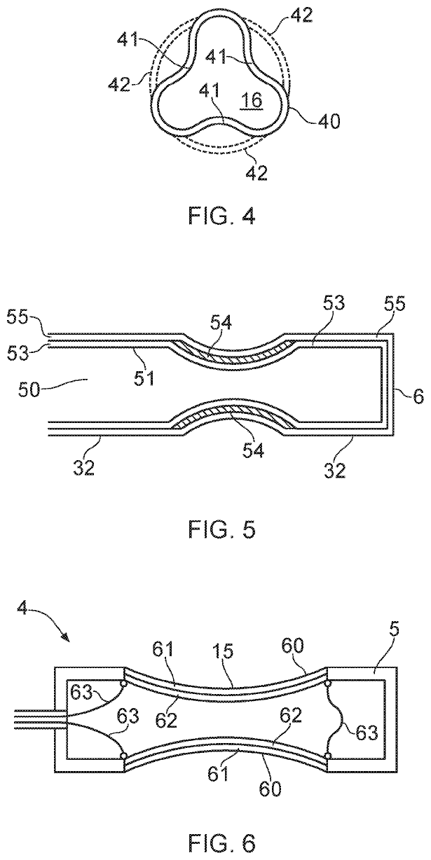

FIG. 4 shows a schematic cross-sectional end view of an alternative spring-loadable mechanical device usable in the balloon of FIGS. 1 and 2;

FIG. 5 shows a schematic cross-sectional side view of a method of manufacturing the spring-loadable mechanical device of FIG. 3;

FIG. 6 shows a schematic cross-sectional side view of another spring-loadable mechanical device usable in the balloon of FIGS. 1 and 2, actuatable by electrical actuation;

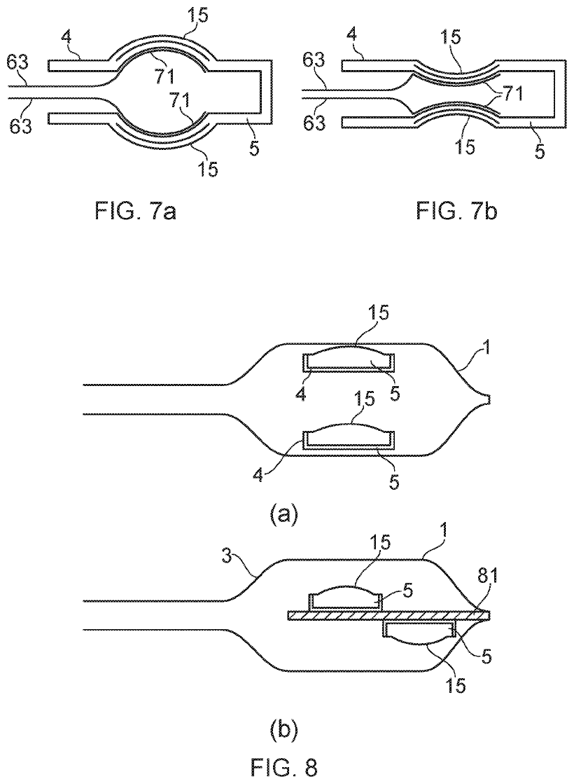

FIG. 7 shows schematic cross-sectional side views of another spring-loadable mechanical device usable in the balloon of FIGS. 1 and 2, thermally actuatable by electrical actuation;

FIG. 8 shows schematic cross-sectional side views of further spring-loadable mechanical devices usable in a balloon with pressure actuation;

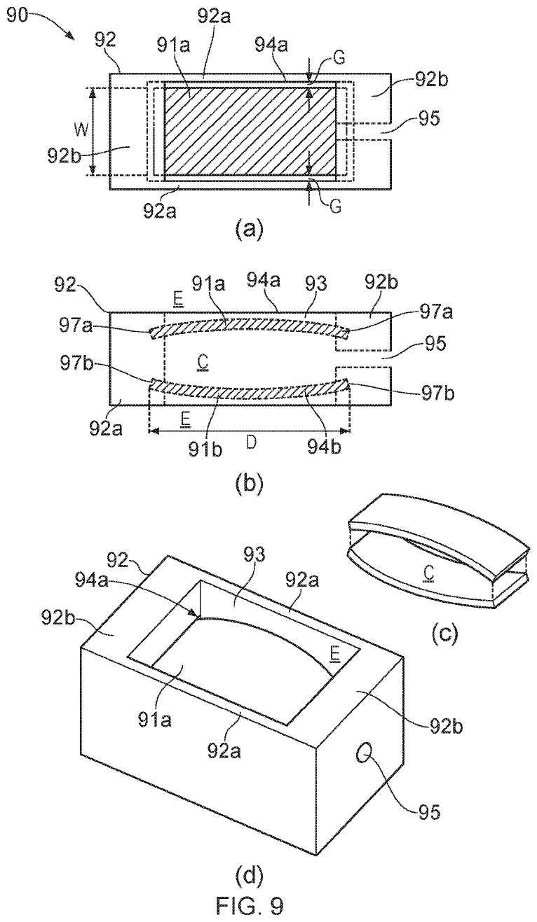

FIG. 9 shows (a) a schematic plan view, (b) a schematic cross-sectional side view, (d) a schematic perspective view of an alternative spring-loadable mechanical device usable in a balloon with pressure actuation; and (c) a perspective view of elastic strips used therein in an expanded (convex) configuration;

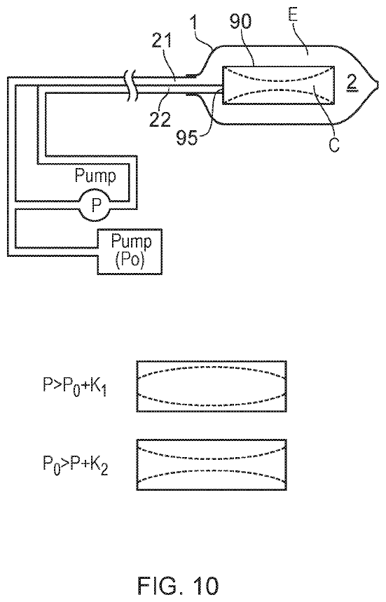

FIG. 10 shows a schematic diagram of a medical device incorporating the spring-loadable mechanical device of FIG. 9.

In contrast to the system described in WO 2017/168145 mentioned above, in the medical device now described herein, percussive pressure impulses within a fluid filling the balloon may be generated within the balloon itself rather than relying on pressure impulses being delivered down a lumen of the catheter. Further, this localised generation of percussive pressure impulses within the fluid filling the balloon can be used to create cavitation bubbles which subsequently collapse. The cavitation bubbles can enhance the percussive effect, as described hereinafter.

With reference to FIG. 1, a balloon 1 which forms part of a medical device is shown in an inflated condition and defines an interior volume 2 which may be filled with saline or other fluid such that the walls 3 of the balloon 1 expand or inflate to reach the walls 10 of a vessel 11 in which the balloon 1 is to be used. The vessel 11 may for example be an artery having a region of calcified material 12 on or within the walls 10 suitable for break-up, disruption or disintegration by the medical device.

Within the interior volume 2 of the balloon 1 is disposed a spring-loadable mechanical structure which is incorporated into a shell 4 comprising a hollow, e.g. tubular, member 5 with a generally or approximately cylindrical form, a closed distal end 6 and a proximal end 7 coupled to a lumen 8 enabling fluid access to an internal volume of the hollow member 5. The hollow member 5 may have any of various tubular cross-sectional profiles including oval or rectangular or other multisided shapes, and may include filleted, chamfered or bevelled edges/corners. In the example shown, the hollow member 5 includes a pair of tensioned ‘popping’ portions or ‘snap-through’ portions 15 which are normally biased to inwardly concave configurations as shown in FIG. 1. As shown more particularly with reference to FIG. 3, the snap-through portions 15 are configured to snap between a first configuration 30 and a second configuration 31. The first configuration is preferably a concave configuration 30 (as viewed from outside the hollow member 5) and the second configuration is preferably a convex configuration 31 (as viewed from outside the hollow member 5). To achieve this functionality, the walls of the hollow member 5 may include thicker, or more rigid, supporting wall portions 32 at axial ends of the hollow member 5 and surrounding thinner or less rigid flexible wall portions 33 defining the snap-through portions 15. The flexible wall portions 33 have a flexibility substantially greater than that of the supporting wall portions.

In this way, the hollow member 5 exemplifies a shell 4 with an internal volume 16 and at least one activation surface 17 provided by the snap-through portions 15. The snap-through portions 15 each exemplify a spring-loadable mechanical structure configured to snap between the first and second configurations when loaded sufficiently from the concave configuration 30 to pass an unstable point from which it snaps or pops through to the convex configuration 31. The operation may also be effected in reverse.

The spring-loadable mechanical structure is thereby configured to store mechanical potential energy during a loading phase while it undergoes compression from the first configuration 30 to an unstable mid-point. The stored mechanical energy can be impulsively released when it is forced to transition to the convex configuration 31. In so doing, the actuation surface 17 locally rapidly compresses fluid in the balloon as the snap-through portion transitions.

The proximal end 9 of the balloon is coupled to a catheter 20, as seen in FIG. 2. In the arrangement of FIGS. 1 to 3, the catheter 20 is preferably one having at least two lumens 21, 22. A first one of the lumens 21 communicates fluid to the internal volume 2 of the balloon 1 from a first fluid source 23 coupled to a distal end of the catheter. The second one of the lumens 22 communicates fluid to the internal volume 16 of the hollow member 5 from a second fluid source 24.

In use, the medical device operates as follows. The interior volume 2 of the balloon 1 is inflated with fluid, such as saline solution, by the first fluid source 23 via a first port 25 that is connected to or forms part of the catheter 20. This enables filling of the balloon 1 to an extent that ensures that the walls 3 of the balloon 1 are in close contact with a calcified region 12 to be treated. Preferably, the balloon 1 is formed from an elastomeric material such that it can conform to the potentially uneven surface area of the calcified region 12 upon inflation of the balloon 1. The hollow member 5 of the shell 4 is filled with fluid via the second lumen 22 using the second fluid source 24 via a second port 26 of the catheter 20. The second fluid source 24 may be a reciprocating pump or other pressure modulating device that applies a fluid with positive and negative pulse pressure, relative to the pressure maintained in the interior volume 2 of the balloon 1.

The first fluid source 23, first lumen 21 and interior volume 2 of the balloon define an outer fluidic system and the second fluid source 24, inner lumen 22 and interior volume 16 of the hollow member 5 define an inner fluidic system.

In one example, the first fluid source 23 may be used to fill the balloon 1 with saline up to a pressure in the range of, e.g. 2 to 20 bar, preferably 2 to 10 bar. The second fluid source 24 may be used to apply positive and negative fluid (e.g. gaseous or liquid) pressures sufficient to overcome the potential energy required to snap the actuation surfaces 17 between the low-energy concave and convex configurations 30, 31.

The snap-through portions 15 are initially deflected inwards in the first configuration 30. As pressure is increased in the interior volume 16, an increasing force is applied to the inner surface of the snap-through portions 15 pushing them towards an unstable median position or mid-point between the first and second configurations 30, 31. Elastic potential energy is stored in the snap-through portions until it is suddenly released when the snap-through portion transitions impulsively to the second (convex) configuration 31. The fast motion of the snap-through portion rapidly displaces fluid in the interior volume 2 of the balloon 1, creating a cavitation bubble or cavitation bubbles. The bubbles collapse and create shock waves carrying significant energy to the walls 3 of the balloon 1. This energy is transferred to the calcified regions 12 and creates cracks that propagate resulting in calcification disruption.

Pulses of positive and negative pressures can be applied repeatedly to the interior volume 16 of the hollow member 5 over a period of time to create repeated pressure pulses until the calcification is broken up.

FIGS. 1 to 3 illustrate a spring-loadable mechanical structure that is embodied in a generally tubular hollow member with a pair of snap-through portions 15. The expression ‘spring-loadable mechanical structure’ as used herein is intended to encompass any mechanical structure comprising an elastically-loadable component capable of storing mechanical energy for impulsive release. Any number of snap-through portions 15 may be envisaged (including one), and these may be distributed circumferentially around the hollow member 5, and/or distributed along the axial length of the hollow member 5. It will also be recognized that many different shapes and configurations of spring-loadable mechanical structure are possible. FIG. 4 illustrates an axial cross-sectional view of a generally triangular cross-section hollow member 40 having three snap-through portions 41 distributed circumferentially around the hollow member 40, each of which can snap between the first (concave) configuration as shown in the drawing to a second (convex) configuration as indicated in dashed outline 42. Other examples may include a flattened tube of rectangular cross-section with snap-through portions on each wide (major) surface of the rectangular cross-section tube.

The spring-loadable mechanical structure may be fabricated from any suitable material or materials. Examples include nickel or nickel-titanium alloy and chromium steel. It is also possible to fabricate the spring-loadable mechanical structure or the snap-through portions 15 of different layers which can have different properties to induce a desired stress in the snap-through portions 15. One example is a bimetallic structure. Another way of fabricating the member is by wafer level microfabrication in silicon, where a cavity and a membrane is created by bonding two wafers and multilayer deposition of e.g. silicon dioxide and silicon nitride on the membrane induces stress.

In the examples described above, the actuation surfaces 17 that create pressure pulses in the saline within the balloon 1 are effectively provided by snap-though portions 15 that are bistable, i.e. each of the convex and concave configurations represents a stable state to which a pressure differential must be applied to push the snap-through portion far enough out of the concave or convex stable configuration towards a median position or mid-point to trigger a transition to the other configuration, e.g. with a series of successive positive and negative differential pressures applied between the volumes 2 and 16. It will be recognized that the snap-through action can also be achieved with a structure that remains biased to one configuration, e.g. the concave configuration 30, which transitions impulsively to the second configuration once the pressure within the hollow member 5 has been increased sufficiently above the pressure in the interior volume 2 of the balloon 1, but will return under its own mechanical spring bias once the pressure in the hollow member 5 has reduced below a certain level. The snap-through portions 15 may therefore exhibit hysteresis in the relationship between their displacement and the applied pressure thereon. The mid-point need not necessarily be half-way in distance between the first and second configurations.

A method for the manufacture of a spring-loadable mechanical structure such as the shell 4 is now described with reference to FIG. 5. A mandrel 50, e.g. formed of aluminium, is fabricated to have a suitable outer shape/profile 51 including suitable concave profiles 52. A first layer 53 of suitable material 53 (e.g. nickel or a nickel alloy such as NiTi) is electroplated (or e.g. vapour deposited) onto the outer surface 51 of the mandrel 50. The first layer 53 of electroplated/deposited material may have a first thickness, e.g. around 50 microns. A layer of screening material 54 is then deposited onto or otherwise covers the concave regions, prior to carrying out a second electroplating/deposition process to deposit a second layer 55 of material. The second layer of electroplated/deposited material 55 may have a thickness of, e.g. around 50 microns, bringing the total thickness up to about 100 microns in the supporting wall regions 32 and closed end 6. The screening material 54 may either prevent or inhibit the formation of a second layer 55 of electroplated material thereover, or may be used as a ‘lift-off’ layer which can be chemically attacked without damaging the electroplated/deposited layers 53, 55 in the supporting wall regions 32 to result in any overlying layer 55 of deposited material to be lifted off the mandrel leaving intact the layer 53 and exposed portions of layer 55. Finally, the mandrel 50 may be etched away leaving the layers 53 and 55 to form the shell 4.

Other microfabrication techniques, such as additive manufacturing ('3D printing') techniques may be used.

The arrangements of medical device described in connection with FIGS. 1 to 4 generally illustrate examples of spring-loadable mechanical structures which can be actuated by an actuation mechanism using fluid pressures transmitted from fluid pressure sources 23, 24 through a pair of catheter lumens 21, 22.

Other examples may use different actuation mechanisms. For example, a mechanical device driven by a control wire passing through a lumen of the catheter 20 may actuate the snap-through portions 5. Such an arrangement could comprise a sliding ring internal to the hollow member 5 which slides axially into position under the control of the control wire. The sliding ring may be disposed in the internal volume 16, in a first axial position inside and aligned with the supporting wall portions 32 and be axially displaceable to a second axial position inside and aligned with the snap-through portions 15 to thereby displace the concave snap-through portions 15 sufficiently until they pop to the convex configuration 31. In this arrangement, the snap-through portions 15 may be snapped back to the concave configuration 30, e.g. by sliding back the sliding ring to its initial position and providing a pressure impulse or other mechanical force to trigger the return of the snap-through portion from convex to concave configuration.

In another example, the actuation mechanism may be provided by an electromechanical device, such as an electromagnetic actuator or solenoid device, which may be positioned within the internal volume 16 and supplied with an electrical control signal via an electrically conductive wire passing along the catheter. It will be understood that use of a mechanical or electromagnetic actuator mechanism means that the catheter 20 may require only one lumen for conveying fluid, together with a control wire or electrical signal wire.

In another example, as now described in connection with FIG. 6, thermal actuation of the actuation surfaces 17/snap-through portions 15 may be used. In this example, each snap-through portion 15 may be formed using a thermally actuatable element 60 such as a bimetallic component. The bimetallic components may each comprise a first metal component layer 61 and a second metal component layer 62 each having a different coefficient of thermal expansion. The bimetallic components 60 may be configured to have a concave configuration as shown in FIG. 6 when at room temperature/body temperature, and to snap-though to a convex configuration when electrically heated to a higher temperature. Electrical heating may be effected by passing current through the bimetallic component 60 from a control wire 63 passing through a lumen 21 of the catheter 20. Instead of direct resistive heating by passage of current through the metal layers themselves, a separate electrical resistor layer may be provided on or in the bimetallic element 60 for indirect resistive heating.

Another way of providing thermal actuation of snap-through portions can be by using a structure such as illustrated in FIGS. 7a and 7b. A bimetallic (or two-layer structure of other materials) component has snap-through portions 15. The two layers of the bimetallic structure have suitable elastic mechanical properties but different thermal expansion coefficients. A combination of fluid pressure application from a second fluid source 24 as seen in FIG. 2 and increasing the temperature of the snap-through portion 15 by a micro heating element 71 deposited, e.g. on its inner surface can produce successive snap-through operations. In such a configuration, the snap-through portions 15 may have one stable state, such as the convex configuration shown in FIG. 7a. By increasing the temperature of the snap-through portion 15 using the heating element 71, a second stable (concave) state can be induced, as seen in FIG. 7b. In this situation, a negative pressure applied by the second fluid source 24 (relative to the balloon pressure) will set the snap-through portion 15 to its concave state. Upon decreasing the temperature (by turning off the micro heating element 71 or otherwise), the temporarily induced stable position (concave) will no longer be stable and the snap-through portion 15 will snap through to its stable state (convex) releasing energy and inducing cavitation bubbles in the interior volume 2 of the balloon 1 (see FIG. 1). Like in the embodiment of FIG. 6, the heating element 71 can be actuated by control wires 63.

In another embodiment as shown in FIG. 8a, the shell 4 defining hollow member 5 with snap-through portions 15 may be hermetically sealed at predetermined pressure relative to that of an inflated balloon 1, such that the snap-through portions 15 are in a convex configuration as shown in the figure. This pressure is large enough to make or keep the concave state unstable. Such hollow members 5 need not be connected to a second fluid source 24 as seen in FIG. 2 and can be disposed on a balloon inner surface or on a structure inside the balloon 1. The snap-through portions 15 may be triggered by increasing the fluid pressure inside the balloon, e.g. using a first fluid source 23 (FIG. 2). In this way, the force exerted by the pressurised fluid source 23 forces the snap-through portions 15 into their concave, second (unstable) configuration. Upon reducing the balloon pressure back to its baseline, the snap-through portions 15 will transit back to their stable (convex) state, releasing energy.

As seen in FIG. 8a, the hollow members 5 may be disposed anywhere within the balloon 1, including at radially outward positions adjacent to or on the balloon walls 3. The snap-through portions 15 may be adjacent to the balloon surface facing radially outward, as in the upper one of the two examples in FIG. 8a, or they may face radially inwards to the interior of the balloon as in the lower one of the two examples. As seen in FIG. 8b, the hollow members 5 may alternatively be disposed on or towards an axially central position, e.g. on a suitable supporting structure 81 within the balloon 1.

Various modifications and adaptations are possible in respect of the embodiments described above.

The snap-through portions 15 can be of any suitable shape and disposition on the hollow member 5, e.g. circular or elliptical shape and disposed in any suitable positions around and along the hollow member to optimise percussive pressure pulses to the wall 3 of the balloon 1. Suitable arrangements of snap-through portions 15 may enable focusing of pulsed energy to specific portions of the balloon wall 3.

The snap-through portions 15 can effectively provide not only an amplification or pressure pulse concentration within the balloon 1 but also focus the impulse energy at specific locations of the balloon wall 3 if desired.

The balloon 1 may be fabricated from an elastic (i.e. stretchable) material which can inflate beyond its relaxed shape and size, or it may be fabricated from a flexible but inelastic (non-stretching) material which can be inflated only up to its maximum size but which may more readily follow contours within the vessel in which it is inflated. The expression ‘balloon’ as used herein is intended to encompass both elastic and inelastic flexible inelastic envelopes which may be filled with a fluid (liquid or gas) such as saline or other medium to inflate the envelope to fit closely within the walls of a vessel 11 such as a coronary artery.

A shape memory material such as nitinol may be used to fabricate the spring-loadable mechanical structure, which may have a memorised configuration and a second configuration to which the structure snaps when electrically heated or otherwise actuated. For example, a nitinol tube could be fabricated to have a suitable shape for a concave/convex transition.

Although the embodiments of FIGS. 1 to 3 have been described in connection with the use of two lumens 21, 22 in the catheter 20 to enable separate pressurization of the interior volume 3 of the balloon 1 and the interior volume 16 of the shell 4, an alternative arrangement using only one lumen is possible by switching the lumen between functions. For instance, the balloon 1 can be first inflated while the lumen is in fluid communication with the interior volume 3 of the balloon. A remotely controlled microvalve in the balloon/catheter interface may then be used to switch the lumen to be in fluid communication with the interior volume 16 of the shell 4, while maintaining the pressure in the balloon. In this way, appropriate pressure changes may then be applied to the spring-loadable mechanical structure of the shell 4.

The illustrated embodiments show snap-through portions 15 that displace in radially inward and radially outward directions from the axis of the balloon 1. However, the device can also be configured to provide snap-through portions or other actuation surfaces 17 that displace in an axial direction, e.g. to provide pressure shock waves travelling to the distal end of a balloon. These may be useful for generating percussive impulses to arteries that are sufficiently occluded to prevent passage of the balloon therethrough

In the illustrated embodiments, the spring-loadable mechanical structure, e.g. shell 4 is generally shown positioned approximately centrally to the interior volume 3 of the balloon 1. However, the structure 4 could be positioned at any location within the balloon 1 to optimise energy focusing. Still further, arrangements of spring-loadable mechanical structures which are reliant on mechanical, electromagnetic or thermal actuation can be positioned on or immediately adjacent to a part of the wall 3 of the balloon for more direct mechanical impact on the calcified material 12 of the vessel 11 in which the balloon is operational. See, for example, an arrangement in FIG. 8a.

A further example of a spring-loadable mechanical structure 90 is shown in FIGS. 9 and 10. FIGS. 9a to 9d illustrate an example of such a structure 90 in which an internal volume of the structure (corresponding to internal volume 16 of the device of FIG. 1) does not need to be hermetically/fluidly isolated from the internal volume of the balloon 1 in which it may be disposed (corresponding to internal volume 2 of FIG. 1).

With reference to FIGS. 9a to 9d, snap-through or popping elements are embodied as elastic strips 91a, 91b made from a suitable alloy or other material which are captured within a housing 92. The housing 92 comprises side walls 92a and end walls 92b together defining an open-sided cavity 93. The end walls 92b each define a pair of slots 97a, 97b which respectively capture respective ends of the strips 91a, 91b. The strips 91a, 91may be pre-formed with a curvature as seen in the cross-sectional view of FIG. 9b or the curvature may be imposed by the length of the strips being slightly greater than the length of the space (distance ‘D’) defined between the respective slots 97a, 97b into which the strips 91a, 91are placed. Thus, when the strips 91a, 91are captured in the housing 92, they will form a concave first configuration (as viewed from outside the housing) or a convex second configuration, as seen in FIGS. 9b, 9c and 9d, similar to the previously described structures.

As shown in FIG. 9a, the width W of the strips 91a, 91is slightly smaller than the width of the openings 94a, 94b defined in the top and bottom aspects of the housing 92 as viewed in FIGS. 9b and 9d. When the strips 91a, 91are captured within the housing 92, there is a small gap G between the respective edges of the strips 91a, 91and the respective housing sidewalls 92a. This gap G creates a clearance large enough to allow free movement of the strips 91a, 91towards and away from the respective openings 94a, 94b but the gap G is small enough to provide a large resistance to fluid flow through the gap. This allows a transient pressure difference to build up between a chamber volume C between the strips 91a, 91and an environment volume E surrounding the structure 90 (e.g. the internal volume 2 of a balloon 1).

The inside surfaces of the housing walls 92a, 92b and the surfaces of the strips 91a, 91b may be coated with a superhydrophobic layer (either partially or fully).

The chamber C that is formed between the strips 91a, 91and the housing walls 92a, 92b is therefore not hermetically sealed or fluid tight, due to the existence of the gaps G. Fluid can also enter the chamber C via an inlet 95 provided by an aperture in one of the walls (e.g. in an end wall 92b as shown in FIGS. 9a, 9b, 9d). Depending on the differential pressure between the chamber C and the environment volume E, fluid can potentially flow out of the chamber C through the small gaps G that show a large resistance to the flow. The resistance to the flow may be enhanced by the surface treatment/coating of the strips 91a, 91and housing wall surfaces using a hydrophobic material.

However, a rapid increase in the pressure inside the chamber C can produce a large differential pressure on the inner sides of the strips 91a, 91during a short time such that there is insufficient time for the fluid to flow through the gaps G and the strips 91a, 91are therefore forced to pop out, i.e. transition from the first (concave) configuration to the second (convex) configuration. Conversely, a rapid decrease in the pressure inside chamber C can apply a force to the outer surfaces of the strips 91a, 91forcing them to pop inwards, i.e. transition from the second (convex) configuration of FIG. 9 to the first (concave) configuration, provided that the pressure external to the chamber C (in environment E) is large enough. Typically, the environment E corresponds to the internal volume of a balloon 1. The pressure changes in the chamber C can be provided via the inlet/aperture 95.

Since the strips 91a, 91are restricted at both ends by the slots 97a, 97b in the housing walls 92b, they undergo snap-through behaviour as described above. The strips 91a, 91b may be able to rotate at their ends (e.g. by loose capture in the slots 97a, 97b) or they may be bonded, welded or otherwise fixed to the housing 92 at their ends.

Upon transitioning from one stable configuration (e.g. concave) to another stable configuration (e.g. convex), the strips 91a, 91release energy stored during the first part of the transition as elastic potential energy during the second part of the transition. The fast popping can cause cavitation in liquid/fluid inside the balloon 1 and/or the liquid can transfer the impact generated by the popping strips 91a, 91b/resulting cavitation to the calcified material 12 on the vessel walls 10 of the vessel 11 in which the device 90 is operating.

FIG. 10 shows an example of use of the spring-loadable mechanical structure 90 of FIG. 9 in operation inside a balloon, similar to that shown in FIG. 2. The pump P0 inflates balloon 1 by pumping a liquid into the balloon and may be operated to maintain a baseline pressure P_{baseline} (e.g. 2-10 bar pressure) in the balloon. A second pump P creates pulsed pressure pulses Pₚᵤₗₛₑ at the fluid inlet 95 rapidly enough and large enough to operate the popping strips 91a, 91as described above. Thus, as Pₚᵤₗₛₑ>P_{baseline}+k1, the strips 91a, 91pop outward to the convex configuration and as P_{baseline}>Pₚᵤₗₛₑ+k2, the strips 91a, 91pop inward to the concave configuration.

The device of FIG. 9 is shown as having two elastic strips 91a, 91defining the chamber C between them, each partially occluding a respective one of the openings 94a, 94b to substantially restrict fluid flow into and out of the cavity via the opening. It will be recognised that the chamber C could alternatively be closed on three sides by fixed walls of the housing 92 and have a single strip (or multiple strips) on one side only adjacent to the opening 94a.

In a general aspect, the housing 92 and strips 91a, 91exemplify a further embodiment of a shell as also exemplified by shell 4 of FIG. 1, and each strip 91a, 91exemplifies an activation surface as previously described. The device 90 may also be configured to operate using thermal actuation (e.g. by electrical heating) as in previously described arrangements. The strips 91a, 91could also be pre-formed such that an automatic return to a stable configuration is effected when a pressure pulse has reduced in magnitude sufficiently to allow the strips 91a, 91to relax. The strips 91a, 91could also be fabricated from a suitable shape memory material. Other adaptations and modes of use as described in connection with FIGS. 1 to 8 may also be applied to the device of FIG. 9.

The arrangements described herein can offer some improvements over the arrangements described in WO 2017/168145 in that the pressure impulses are generated in/at the balloon location and therefore close to the calcification site. The pressure impulses are not therefore susceptible to attenuation by any compliance of the catheter lumens which may otherwise diminish the pressure impulse magnitude or intensity between a pressure source 24 at the proximal end of the catheter 20 and the balloon 1 at the distal end of the catheter.

Other embodiments are intentionally within the scope of the accompanying claims.

1. A medical device comprising:
a balloon having an expandable interior volume for coupling to the distal end of a catheter for a lumen of the catheter to be in fluid communication with the expandable interior volume of the balloon; and
a spring-loadable mechanical structure within the balloon configured snap between first and second configurations upon actuation by an actuation mechanism.

2. The medical device of claim 1 further comprising a catheter having a lumen extending between a distal end and a proximal end of the catheter, the balloon being coupled to the distal end of the catheter and having its interior volume in fluid communication with the lumen of the catheter.

3. The medical device of claim 2 in which the actuation mechanism comprises fluid pressure transmitted through the catheter.

4. The medical device of claim 3 further including a shell disposed within the balloon, the shell defining an internal volume and an activation surface forming part of the shell, the activation surface comprising said spring-loadable mechanical structure configured to snap between said first and second configurations.

5. The medical device of claim 4 in which the first configuration of the activation surface is a concave configuration and the second configuration is a convex configuration.

6. The medical device of claim 4 in which the lumen comprises a first lumen communicating with the interior volume of the balloon and a second lumen communicating with the internal volume of the shell.

7. The medical device of claim 1 in which: (a) the spring-loadable mechanical structure s configured to store mechanical potential energy during a loading phase while it goes from a first configuration to an unstable mid-point which is impulsively released when the mechanical structure is forced to transition to the second configuration, (b) the actuation mechanism comprises an electromechanical device; or (c), the spring-loaded mechanical structure comprises a thermally actuatable element.

8. The medical device of claim 6 further including a means for applying fluid pressure to the interior volume of the balloon through the first lumen and a means for applying pressure pulses to the internal volume of the shell through the second lumen.

9. The medical device of claim 4 in which the shell comprises a hollow ber with supporting wall portions and at least one flexible wall portion supported by the supporting wall portions, the flexible wall portion having a flexibility substantially greater than the supporting wall portions and configured to snap between the first and second configurations.

10. The medical device of claim 9 in which the supporting wall portions have a first thickness and the flexible wall portions have a second thickness less than the first thickness.

11. The medical device of claim 9 in which at least the spring-loadable mechanical structure is formed of nickel, nickel alloy or of nickel-titanium alloy.

12. (canceled)

13. The medical device of claim 12 in which the electromechanical device is located within the balloon and is coupled to a control wire extending along a catheter to the balloon.

14. (canceled)

15. The medical device of claim 7 in which the spring-loadable mechanical structure comprises a bimetallic element

16. The medical device of claim 14 in which the actuation mechanism comprises an electrical heating element.

17. The medical device of claim 16 in which the electrical heating element forms part of the bimetallic element.

18. The medical device of claim 1 in which the spring-loadable mechanical structure within the balloon comprises a housing defining a cavity with at least one opening to the cavity and an elastic strip retained within the cavity and partially occluding the cavity opening to substantially restrict fluid flow into and out of the cavity via the opening,
wherein the elastic strip defines an activation surface configured to snap between first and second configurations, the first configuration comprising a concave configuration and the second configuration comprising a convex configuration.

19. The medical device of claim 18 further comprising an aperture in a wall of the housing, the aperture extending to the cavity to enable fluid pressure pulses to be delivered from outside the housing into the cavity to actuate the elastic strips between the first and second configurations.

20. A method of breaking up, disrupting or disintegrating calcified or other hardened material within vessels of the human or animal body comprising:
using a catheter to deploy a medical device into the vessel, the medical device comprising a balloon having an expandable interior volume coupled to the distal end of the catheter such that a lumen of the catheter is in fluid communication with the expandable interior volume of the balloon:
inflating the balloon within the vessel with a fluid to fill e expandable interior volume of the balloon; and
actuating a spring-loadable mechanical structure within he balloon to snap between a first configuration and a second configuration using an actuation mechanism, to thereby create a pressure impulse in the fluid within the interior volume of the balloon.

21. The method of claim 19 in which the pressure impulse generates cavitation in the fluid within the balloon.
